(19) United States
(12) Patent Application Publication
(10) Pub. No.: US 2019/0083817 A1
(43) Pub. Date: Mar. 21, 2019

(54) SYSTEMS, METHODS, AND COMPUTER READABLE MEDIA FOR ISCHEMIC INJURY PROTECTIVE ULTRASOUND

(71) Applicant: University of Virginia Patent Foundation, Charlottesville, VA (US)

(72) Inventors: Mark D. Okusa, Charlottesville, VA (US); Joseph C. Gigliotti, Ruckersville, VA (US); John A. Hossack, Charlottesville, VA (US)

(21) Appl. No.: 16/097,024

(22) PCT Filed: May 3, 2017
(86) PCT No.: PCT/US17/30893

§ 371 Date: Oct. 26, 2018

Related U.S. Application Data
(60) Provisional application No. 62/331,104, filed on May 3, 2016.

(51) Int. Cl. A61N 7/00

(52) U.S. Cl. CPC A61N 7/00; A61N 2007/0004; A61N 2007/0052

(57) ABSTRACT

Disclosed are methods, systems, and non-transitory computer readable media having stored thereon executable instructions that when executed by the processor of a computer control the computer to perform therapeutic ultrasound, such for the treatment of Ischemic reperfusion injury (IRI). The methods, systems, and non-transitory computer readable media can involve steps comprising: imaging a target tissue region within said subject, such as wherein the target tissue region comprises spleen tissue; identifying a volume region of interest (ROI) in said target tissue; and applying ultrasonic energy to the ROI, wherein applying the ultrasonic energy comprises emitting a sequence of ultrasonic pulses from an ultrasound transducer, the sequence of ultrasound pulses having predetermined frequency, mechanical index, pulse lengths, and pulse spacings, while performing a volumetric sweep through the ROI systematically for a selected total time duration, such as between about 3 minutes and about 15 minutes.

### CROSS-REFERENCE TO RELATED APPLICATIONS

This application claims the benefit of U.S. Provisional Patent Application Ser. No. 62/331,104, filed May 3, 2016, the disclosure of which is incorporated herein by reference in its entirety.

### TECHNICAL FIELD

The presently disclosed subject matter relates to methods, systems, and computer readable media for therapeutic use of ultrasound. In some embodiments, the methods, systems, and computer readable media for therapeutic ultrasound provide for the protection of tissues from ischemia-reperfusion injury.

### BACKGROUND

Acute Kidney Injury (AKI) is a significant clinical problem. Acute kidney injury resulting from hypotension, ischemia-reperfusion (IR), nephrotoxins such as radiocontrast agents, and sepsis to name a few causes, is an abrupt cessation of kidney function leading to fluid overload, electrolyte acid-base imbalance and inability to rid the body of metabolic toxins; in severe cases it leads to death (FIG. 1). Therefore, prevention and treatment of AKI is an important clinical problem, as mortality in patients with AKI, especially in critically ill patients, remains alarmingly high (40-60% of ICU patients) despite substantial advances in techniques of resuscitation and renal replacement therapy. Over a 15-yr period (1988-2002) the incidence of AKI continued to increase; there were 1,083,745 discharges with AKI. The greatest predisposing factor to the development of AKI is chronic kidney disease (CKD). There are 20-40 million people in the USA with CKD and they have a high risk factor for sustaining AKI. Currently there are no FDA approved drugs for AKI. Small sample size, poorly timed administration during trials, and adverse effects associated with drugs have hampered the success of clinical trials.

The pathogenesis is complex. Following ischemia-reperfusion injury (IRI) there are alterations in renal medullary vascular tone. Medullary vascular congestion due to leukostasis contributes to a decrease in blood flow. The cascade of events following IR leading to endothelial cell dysfunction and activation of tissue-resident and infiltrating includes the coordinated action of cytokines/chemokines, reactive oxygen intermediates and adhesion molecules. Modulation of central and peripheral neural activity to target organ function has gained considerable interest recently. Current techniques for modulating neural activity include pharmacological agents or mechanical devices. Surgical and invasive procedures are required for most pharmacological agents that require direct application and for mechanical devices. Thus, additional approaches for treating AKI, and other related diseases or disorders with similarly complex pathogenesis, represent a long-felt and ongoing need in the art. For example, ischemic-reperfusion injuries can occur in other organ systems (such as the heart), and methods described in the context of AKI may be used in these other applications.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method of protecting a subject in need thereof from ischemia/reperfusion injury (IRI) and/or mitigating in a subject an inflammatory condition, disease or disorder, the method comprising: imaging a target tissue region within said subject, wherein the target tissue region comprises spleen tissue; identifying a volume region of interest (ROI) in said target tissue; and applying ultrasonic energy to the ROI, wherein applying the ultrasonic energy comprises emitting a sequence of ultrasonic pulses from an ultrasound transducer, the sequence of ultrasound pulses having predetermined frequency, mechanical index, pulse length, and pulse spacing, while performing a volumetric sweep through the ROI systematically for a selected total time duration of between about 3 minutes and about 15 minutes. In some embodiments, the total time duration is about 10 minutes.

In some embodiments, the ultrasonic energy has a frequency of between about 1 and about 10 megahertz (MHz). In some embodiments, the sequence of ultrasound pulses comprises burst pulses having a frequency of about 1.7 MHz and imaging pulses having a frequency of about 3 MHz. In some embodiments, the ultrasonic energy is applied at a mechanical index ranging from about 0.5 to about 1.9.

In some embodiments, the sequence of ultrasound pulses comprises a series of burst pulse sequences, optionally wherein the duration of each burst pulse sequence is between about 3 and about 5 seconds and the time interval between burst pulse sequences is about 1 second.

In some embodiments, the ultrasonic energy “on” time, relative to the time between successive pulses, lies in the range of about 1% to about 10%. In some embodiments, each burst pulse sequence comprises a series of pulse sub-sequences, optionally wherein each pulse sub-sequence comprises a series of pulse repetitions at a single lateral location.

In some embodiments, the method protects a subject in need thereof from kidney IRI. In some embodiments, the subject is a human. In some embodiments, the ultrasonic energy is applied about 24 to 48 hours prior to the subject undergoing a surgery that will result in an ischemic event.

In some embodiments, the presently disclosed subject matter provides a system for effecting ultrasound-based protection from ischemia/reperfusion injury (IRI) and/or mitigating in the subject an inflammatory condition, disease or disorder, said system comprising: an imaging device for imaging a target tissue region; a transducer for performing a volumetric sweep through the target volume systematically for a selected total time duration and for applying ultrasonic energy to a volume region of interest (ROI), wherein applying the ultrasonic energy comprises emitting a sequence of ultrasonic pulses from the transducer, the sequence of ultrasound pulses having predetermined frequency, mechanical index, pulse length(s), and pulse spacing(s), while performing a volumetric sweep through the ROI systematically; a processor for identifying a volume ROI by highlighting a region on one of more image frames as the transducer is swept through a volume; and a controller for selecting a therapeutic ultrasound dose comprising a predetermined frequency, mechanical index, pulse length, and pulse spacing. In some embodiments, the imaging device and the transducer are the same component.

In some embodiments, said transducer is placed in a mechanical translation stage to automatically effect the sweep. In some embodiments, said transducer is configured to track elevation motion to assure that a desired sweep velocity is used. In some embodiments, said transducer is a sector transducer array, optionally wherein it steers to +/−45 degrees. In some embodiments, said transducer is a curved linear transducer array. In some embodiments, said transducer comprises a 2D array that can sweep through an entire target volume without physical translation.

In some embodiments, said selected total time duration ranges from about 3 minutes to about 15 minutes, optionally wherein said total time duration is about 10 minutes. In some embodiments, the ultrasonic energy has a frequency of between about 1 and about 10 megahertz (MHz). In some embodiments, the sequence of ultrasound pulses comprises burst pulses having a frequency of about 1.7 MHz and imaging pulses having a frequency of about 3 MHz. In some embodiments, the ultrasonic energy is applied at a mechanical index ranging from about 0.5 to about 1.9.

In some embodiments, the sequence of ultrasound pulses comprises a series of burst pulse sequences, optionally wherein the duration of each burst pulse sequence is between about 3 and about 5 seconds and the time interval between burst pulse sequences is about 1 second.

In some embodiments, the ultrasonic energy “on” time, relative to the time between successive pulses, lies in the range of about 1% to about 10%. In some embodiments, each burst pulse sequence comprises a series of pulse sub-sequences, optionally wherein each pulse sub-sequence comprises a series of pulse repetitions at a single lateral location.

In some embodiments, said system uses rates of image decorrelation to estimate rate of elevational motion and to provide feedback as to “too fast” or “too slow”. In some embodiments, said system measures instantaneous sweep velocity and provides an audible or visual cue as to “too fast” or “too slow”.

In some embodiments, said system further comprises an image library database. In some embodiments, said image library data base provides an image comparison/similarity algorithm that is used to automatically identify spleen and thereby identify an optimal ROI that encompasses it.

In some embodiments, the presently disclosed subject matter provides a non-transitory computer readable medium having stored thereon executable instructions that when executed by the processor of a computer control the computer to perform steps comprising: imaging a target tissue region within said subject, wherein the target tissue region comprises spleen tissue; identifying a volume region of interest (ROI) in said target tissue; and applying ultrasonic energy to the ROI, wherein applying the ultrasonic energy comprises emitting a sequence of ultrasonic pulses from an ultrasound transducer, the sequence of ultrasound pulses having predetermined frequency, mechanical index, pulse length, and pulse spacing, while performing a volumetric sweep through the ROI systematically for a selected total time duration of between about 3 minutes and about 15 minutes. In some embodiments, the total time duration is about 10 minutes.

In some embodiments, the ultrasonic energy has a frequency of between about 1 and about 10 megahertz (MHz). In some embodiments, the sequence of ultrasound pulses comprises burst pulses having a frequency of about 1.7 MHz and imaging pulses having a frequency of about 3 MHz. In some embodiments, the ultrasonic energy is applied at a mechanical index of ranging from 0.5 to about 1.9.

In some embodiments, the sequence of ultrasound pulses comprises a series of burst pulse sequences, optionally wherein the duration of each burst pulse sequence is between about 3 and about 5 seconds and the time interval between burst pulse sequences is about 1 second.

In some embodiments, the ultrasonic energy “on” time, relative to the time between successive pulses, lies in the range of about 1% to about 10%. In some embodiments, each burst pulse sequence comprises a series of pulse sub-sequences, optionally wherein each pulse sub-sequence comprises a series of pulse repetitions at a single lateral location.

In some embodiments, the executable instructions that when executed by the processor of a computer control the computer to interact with an image library database. In some embodiments, the executable instructions comprise an image comparison/similarity algorithm that is used to automatically identify spleen and thereby identify an optimal ROI that encompasses it.

Accordingly, it is an object of the presently disclosed subject matter to provide methods, systems, and computer readable media for therapeutic ultrasound.

An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIG. 1 is a schematic representation showing that cholinergic anti-inflammatory pathway is central to various experimental models of inflammatory disease.

FIG. 2 is a sample spleen image showing sonographic measurement of the spleen.

FIG. 3 is a sample spleen image with a region of interest (ROI) approximately encompassing the target region.

FIG. 4 is a schematic representation of an ultrasound system 700 according to an aspect of an embodiment of the presently disclosed subject matter, which is referred to in order to generally describe the operations of an ultrasound system to produce an image of an object 13.

FIG. 5 is a block diagram illustrating an example of a machine 400 upon which one or more aspects of embodiments of the presently disclosed subject matter (e.g., discussed methodologies) can be implemented (e.g., run).

FIG. 6 is a schematic diagram of ultrasound (US) sequencing employed in Example 2.

FIG. 7 is a schematic diagram of ultrasound (US) sequencing employed in Example 2.

FIGS. 8A and 8B are plots of a single imaging pulse and of an imaging pulse train, respectively; and FIG. 8C is a photograph of a related ultrasound system display. The imaging pulse frequency is 2.8 Megahertz (MHz); the interval between pulses is approximately 200 microseconds (μs); and focal depth is set at 60 millimeters (mm).

FIGS. 9A and 9B are a plot and a photograph of an ultrasound system display, respectively, showing a single burst pulse. Burst pulse frequency is: 1.7 Megahertz (MHz) and the burst train duration was set to 50 microseconds (μs), continuously repeated at an interval of 1 second (s) using a programmable mode as described in Example 2). The lowest interval possible is 1 s and the lowest duration possible is 50 μs. Interval between pulses is approximately 6 ms.

FIGS. 10A-10C are a series of photographs of an ultrasound system display showing a burst frame with an interval between A-lines of 6 milliseconds (ms) (FIG. 10A); a single burst A-line, with an interval between pulses of 0.3 ms (FIG. 10B); and a single burst pulse (FIG. 10C).

FIGS. 11A-11C are a series of plots showing the ultrasound settings employed in Example 2: del T (interval between burst frames) set to 54 milliseconds (ms) (FIG. 11A); burst A line pulse interval=0.3 ms=3.3 kilohertz (KHz); amplitude variation in burst A line reflects off axis pulses (FIGS. 11B and 11C).

FIGS. 12A and 12B are a set of plots showing burst frame trains of varying durations as employed in Example 2 (i.e., 49 milliseconds (ms) in FIG. 12A and 147 ms in the three frame burst train of FIG. 12B).

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

In some embodiments, the presently disclosed subject matter provides an ultrasound system (and related method) comprising an optimized ultrasound scanner and an appropriate transducer that performs the dual function of imaging a target tissue, such as the spleen, and then administering a precisely and accurately controlled therapeutic ultrasound treatment sequence.

An aspect of an embodiment of the presently disclosed subject matter provides, but is not limited to, an ultrasound approach that is entirely non-invasive.

An aspect of an embodiment of the presently disclosed subject matter provides, but is not limited to, an ultrasound approach whereby microbubbles or any other drug, contrast agent, or other matter are not required to be injected.

An aspect of an embodiment of the presently disclosed subject matter provides, but is not limited to, an ultrasound imaging sequence comprising regular B-Mode (i.e., Brightness mode) imaging.

An aspect of an embodiment of the presently disclosed subject matter provides, but is not limited to, a therapeutic ultrasound system (and related method) that comprises a defined sequence of ultrasound pulses falling within an identified range of amplitude, pulse duration and total pulsing duration. For example, particular sequences have therapeutic effect when applied methodically in accordance with aspects of embodiments of the presently disclosed subject matter.

An aspect of an embodiment of the presently disclosed subject matter provides an ultrasound system (and related method) comprising a specially programmed ultrasound scanner and an appropriate (imaging+therapy capable) transducer, that performs the dual function of imaging the spleen (or other tissue) and then administering a precisely and accurately controlled therapeutic ultrasound treatment sequence. In some embodiments, the approach is non-invasive. In some embodiments, an invasive approach, such as but not limited to using catheter based ultrasound, can be employed. The intensities and durations of ultrasound are believed to be safe and produce zero, or at most very limited, off-target changes in tissue form or function.

An aspect of an embodiment of the presently disclosed subject matter provides, but is not limited to, an improved role in each of disease diagnosis and disease treatment.

An aspect of an embodiment of the presently disclosed subject matter provides, but is not limited to, the use of ultrasound applied to a specific organ (for example, the spleen, but not limited thereto) so as to effect a therapeutic effect through a newly identified pathway (such as the cholinergic anti-inflammatory pathway)—i.e., the ensonification of the tissue gives rise to a newly identified therapeutically desirable pathway.

An aspect of an embodiment of the presently disclosed subject matter provides, but is not limited thereto, an approach to imaging a patient experiencing ischemia/reperfusion injury (IRI), a patient who could face IRI in near future, such as a patient anticipating a surgical procedure, and/or a patient in need of mitigation of an inflammatory condition, disease or disorder and then administering, in an image-guided manner, ultrasound energy, of sufficient intensity and duration, that achieves an IRI protective effect, an IRI preventative effect, and/or an inflammation mitigation effect.

An aspect of an embodiment of the presently disclosed subject matter provides an ultrasound system (and related method) comprising an optimized ultrasound scanner and an appropriate transducer that performs the dual function of imaging the spleen and then administering a precisely and accurately controlled therapeutic ultrasound treatment sequence.

### I. DEFINITIONS

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising,” which is synonymous with “including,” “containing,” or “characterized by,” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Unless otherwise indicated, all numbers expressing quantities of temperature, time, concentration, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes, but is not limited to, 1, 1.5, 2, 2.75, 3, 3.90, 4, and 5).

The term “treatment” as used herein refers to any treatment that has a beneficial effect in treatment of a disease or disorder. Thus, a treatment can include administration of an effective amount of US.

“Co-administer” can include simultaneous and/or sequential administration of two or more treatments.

The term “effective amount” can refer to an amount sufficient to produce a selected effect, such as alleviating or preventing symptoms of a disease or disorder.

The term “more effective” means that the selected effect is alleviated to a greater extent by one treatment relative to the second treatment to which it is being compared.

The term “concurrently” when used in the context of the administration of two or more different treatments can refer to the simultaneous administration of the two or more treatments (e.g., the administration of the two or more treatments roughly the same time (e.g., within about 24 hours or less, about 12 hours or less, about 6 hours or less, about 4, hours or less, about 2 hours or less, or about 30 minutes or less). However, “concurrently” can also be used to refer to the administration of two or more treatments at different times, but while at least one of the treatments is ongoing.

Thus, as used herein, “prior to” in the context of the administration of one or more treatments can refer to a situation where a subject has already received a full course of one treatment before a second treatment is initiated.

Acoustic power is expressed in a variety of ways by those skilled in the art. One method of estimating the acoustic power of an acoustic wave on tissue is the “Mechanical Index”. The Mechanical Index (MI) is a standard measure of the acoustic output in a diagnostic ultrasound system, defined as the peak negative pressure, or rarefactional pressure, (in units of MPa—megaPascals) of an ultrasound wave propagating in a uniform medium, divided by the square root of the centre frequency (in units of MHz) of the transmitted ultrasound pulse. The uniform medium is assumed to have an attenuation of 0.3 dB/cm/MHz (attenuation coefficient divided by ultrasound frequency). MI is proportional to pulse voltage and inversely proportional to the square root of the pulse frequency. The mechanical index (MI) is intended to offer an approximate guide to the likelihood of the occurrence of cavitational-related bioeffects. High acoustic pressure ultrasound systems generally have a MI greater than 10. Low acoustic pressure systems generally have a MI lower than 5. For example, diagnostic ultrasound systems are limited by U.S. FDA to a Mechanical Index not to exceed 1.9.

Another measurement used by those skilled in the art is the spatial peak, peak average intensity (“Isppa”). The intensity of an ultrasound beam is greater at the center of its cross section than at the periphery. Similarly, the intensity varies over a given pulse of ultrasound energy. Isppa is measured at the location where intensity is maximum averaged over the pulse duration. Isppa for high acoustic pressure or HIFU applications ranges from approximately 1500 W/cm² to 9000 W/cm². Diagnostic ultrasound equipment, for instance, will generally have, and an Isppa less than 700 W/cm².

Yet another way in which ultrasound waves can be characterized is by the amplitude of their peak negative pressure. “High acoustic pressure” or “HIFU” applications employ waves with peak amplitudes in excess of 10 MPa. “Low acoustic pressure” ultrasound includes ultrasound waves will generally have peak negative pressures in the range of 0.01 to 5.0 MPa. Diagnostic ultrasound equipment, for example, will generally have a peak amplitude less than 3.0 MPa.

Both high and low acoustic pressure ultrasound systems generally operate within the frequency range of 250 KHz-10.0 MHz. Diagnostic imaging typically uses frequencies of about 1.0 MHz to about 5.0 MHz. One known low acoustic pressure ultrasound probe may produce ultrasound having a frequency as high as 7.5 MHz. Some low acoustic pressure ultrasound probes may produce ultrasound frequencies as high as 10.0 MHz. Physical therapy ultrasound systems generally operate at frequencies of either 1.0 MHz or 3.3 MHz.

### II. IMAGE GUIDED ULTRASOUND FOR PROTECTION FROM ISCHEMIA REPERFUSION INJURY

Ultrasound (US) is a tool that can achieve spatial focused delivery of energy. Among its non-thermal properties, US can also modulate frog peripheral nerve activity and stimulate electrical activity in hippocampal neurons by activating voltage-gated ion channels, and transcranial US-induced neuromodulation produced limb movement in rodents. These findings suggest a potentially important, unrecognized, innovative therapeutic approach to human disease based on biomechanical non-thermal effects of US.

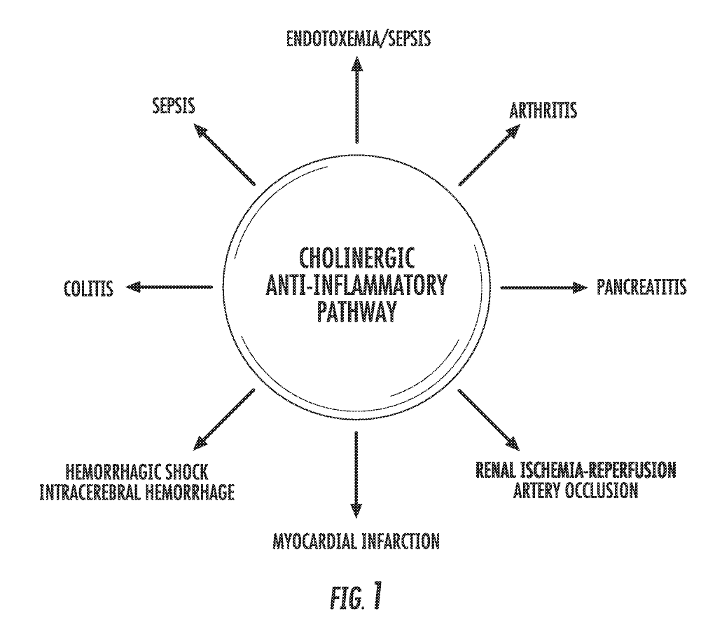

In accordance with the presently disclosed subject matter, it has been found that US pulses protect the kidney from ischemia-reperfusion injury (IRI) in an animal model of AKI. Without being bound to any one theory, it is believed that US protection is afforded because US can activate the cholinergic anti-inflammatory pathway (CAP). The CAP serves as an interface between the nervous and immune systems to maintain homeostatic mechanisms to respond to stress or injury and has been implicated as a favorable therapeutic target in numerous diseases/disorders, such as, but not limited to, sepsis, colitis, arthritis, endotoxemia, pancreatitis, hemorrhagic shock, intracerebral hemorrhage, myocarcial infarction (MI), and renal ischemia-reperfusion injury. See FIG. 1. The rapid response facilitated by nerve conduction provides immediate signaling to attenuate inflammation. CAP involves the spleen, as it appears to play a role in an inflammatory reflex for the neural control of inflammation and provides a potential therapeutic target for immune-mediated diseases.

In some embodiments, the presently disclosed subject matter provides a method of protecting a subject in need thereof from ischemia/reperfusion injury (IRI). In some embodiments, the method comprises: imaging a target tissue region within said subject; identifying a volume region of interest (ROI) in said target tissue; applying ultrasonic energy to the ROI, wherein applying the ultrasonic energy comprises emitting a sequence of ultrasonic pulses from an ultrasound transducer. In some embodiments, the sequence of ultrasound pulses has a predetermined frequency, mechanical index, pulse lengths, and pulse spacings. In some embodiments, the sequence of ultrasonic pulses is emitted while performing a volumetric sweep through the ROI systematically for a selected total time duration. In some embodiments, the total time duration ranges from about 3 minutes to about 15 minutes. In some embodiments, the total time duration ranges from about 5 minutes to about 15 minutes. In some embodiments, the total time duration is about 10 minutes.

In some embodiments of the presently disclosed subject matter, a largely unmodified diagnostic ultrasound scanner is employed. In some embodiments, an unmodified sector scanning transducer (e.g. Siemens 4V2 or 4V1, Siemens Medical Solutions USA, Inc., Mountain View, Calif., United States of America, or GE/Philips equivalents, GE Healthcare, Chicago, Ill., United States of America; Philips, Amsterdam, Netherlands) can be implemented as well. An aspect of an embodiment of the present presently disclosed subject matter provides, but is not limited thereto, a user interface to guide the imaging and therapeutic operations. Initially, the spleen is detected in the ultrasound image and then a volumetric scan sequence is identified.

In some embodiments, the target tissue region comprises spleen tissue. Furthermore, in some embodiments of the presently disclosed systems and methods are used, but not limited thereto, with regard to the following clinical applications: therapy for AKI; therapy for protection from IRI post myocardial infarction; therapy for protection from IRI in connection with surgical procedures; therapy for colitis; therapy for endotoxemia/sepsis; therapy for pancreatitis; and therapy for rheumatoid arthritis. In some embodiments, the subject of the presently disclosed methods is typically mammalian. For example, subjects of the presently disclosed subject matter include, but are not limited to, humans and other primates, as well as other mammals including commercially relevant mammals or mammals kept as pets or in zoos, such as cattle, pigs, horses, sheep, cats, dogs, goats, bears, rabbits and rodents. Suitable subjects also include birds, including commercially relevant birds and birds kept as pets, such as chickens, ducks, geese, parrots, and turkeys. In some embodiments, the subject is a human. In some embodiments, the ultrasonic energy is applied about 24 to 48 hours prior to the subject undergoing a surgery that will result in an ischemic event. In some embodiments, the subject is in need of mitigation of an inflammatory condition, disease or disorder, such as but not limited colitis, endotoxemia/sepsis, pancreatitis, or rheumatoid arthritis.

According to some embodiments of the presently disclosed subject matter, a patient is diagnosed or otherwise screened in advance for IRI protection therapy. For example, patients scheduled for certain types of invasive surgeries and patients in the intensive care unit (ICU) with IRI risk (e.g., ICU patients with multi-organ disease and/or sepsis) are identified. B-mode ultrasound is used to find the spleen using well-known and documented anatomical landmarks. As previously described (see, e.g., J Ultrasound Med., 2011, 30:1281-1293), the spleen can be best examined with the patient in a supine or lateral recumbent position, while the patient exhales so that the upper pole of the spleen is not covered by lung tissue, using the 10^{th} and 11^{th} intercoastal spaces as the acoustic window for ultrasound transmission with sonographic examination from the diaphragmatic end to the lower pole, and/or using a curved array transducer with a median frequency of 3 to 5 MHz. A normal adult human spleen is about 10 to 12 centimeters (cm) in length. See J. Ultrasound Med., 2011, 30:1281-1293. The ultrasound operator then determines the optimal format of volumetric scan—i.e. the orientation of the imaging/therapy plane and how that plane will be translated to encompass a volume ROI.

Using the ultrasound system user interface, the ultrasound operator can enter desired therapy parameters: frequency, pulse intensity, interval, etc. Suitable parameters are described in more detail hereinbelow and also include the following. In some embodiments, the ultrasonic energy has a frequency of between about 1 and about 10 megahertz (MHz). In some embodiments, sequence of ultrasound pulses comprises burst pulses (e.g., having a frequency of about 1.7 MHz) and imaging pulses (e.g., having a frequency of about 3 MHz). In some embodiments, ultrasonic energy is applied at a mechanical index ranging from about 0.5 to about 1.9, such as about 1, 1.2 or 1.7.

In some embodiments, the sequence of ultrasound pulses comprises a series of burst pulse sequences, wherein the duration of each burst pulse sequence is between about 3 and about 5 seconds and the time interval between burst pulse sequences is about 1 second. In some embodiments, each burst pulse sequence comprises a series of pulse sub-sequences. In some embodiments, each pulse sub-sequence can be referred to as a “burst frame”. In some embodiments, each pulse sub-sequence has a duration of about 50 microseconds or greater, such as about 54 microseconds. In some embodiments, each pulse sub-sequence has a duration of about 50 milliseconds or greater, such as about 54 milliseconds. In some embodiments, one or more (or each) pulse sub-sequence comprises a series of pulse repetitions at a single lateral location (e.g., within the ROI).

Other forms of pulse design are possible. For example, in some embodiments, the ultrasonic energy “on” time, relative to the time between successive pulses, lies in the range of about 1% to about 10%. That is, pulses can have an “on” time, relative to pulse interval (i.e. “duty cycle”), lying in the range of 1 to 10%, e.g. 1%, 2%, 3%, 4%, 5%, 6%, 7%, 8%, 9%, or 10%. For example, a 5 cycle 2 MHz pulse is 2.5 microseconds long. At 1% duty cycle, the inter pulse interval is 250 microseconds. At 10% duty cycle, the inter pulse interval is 25 microseconds.

The ultrasound operator can also define a range of motion to fully encompass the spleen. For example, if the spleen fits entirely within the image plane of the transducer, then the transducer can be translated, or tilted, to encompass the 3D spleen volume. If the spleen does not fit within the image plane of the transducer there are two solutions. First, the transducer can be rotated so as to acquire a “short axis” as opposed to a “long axis”. Once the short axis is acquired, the transducer can be swept elevationally to encompass the 3D spleen volume. Alternatively, the ultrasound operator can select a transducer with a larger field of view. Typically, a large curved linear transducer will have the greatest near field and far field image dimension and can practically assure that the spleen can be fully encompassed within a volumetric sweep.

Next, the ultrasound operator can perform a “dry run”. In the “dry run,” the ultrasound machine, in diagnostic imaging mode only, scans across the tissue region and color codes (translucent overlay) the region that would be insonified with a “live” therapy insonification. The scanner produces a three dimensional (3D) representation of the anatomy and the color overlay. If the color overlay correctly insonifies all the tissue required, the ultrasound operator hits a “go” button and therapy is administered. If the overlap is incorrect, the operator modifies the scanning orientation and position and repeats the process until correct overlay is obtained and a “live” procedure can be conducted.

The volumetric scan involves sweeping the transducer through a volume—i.e. stepping the transducer gradually in the direction orthogonal to the imaging plane. The volumetric scan can use a computer controlled motion stage or some form of 3D positioning system that allows for freehand sweep. Freehand sweeping can be performed using optical tracking, magnetic tracking, or image registration-based tracking. Image registration-based tracking can be convenient because it is unobtrusive, but does involve a minor transducer modification, such as, but not limited to, the “I-Beam” approach described in Hossack et al., IEEE Transactions on Ultrasonics Ferroelectrics & Frequency Control, 2002, 49(8):1029-1038. The “I-Beam” has a central (conventional) imaging array. Two perpendicularly oriented “tracking” arrays are attached to either end of the central imaging array (so as to form a shape “I”.) The “tracking” arrays are also imaging arrays—forming images perpendicular to that obtained by the central array. As the central array image plane is swept across a volume it collects a sequence of 2D image slices arranged in a “breadslice” manner. By tracking the motion increments detected on the “tracking” array acquired images (e.g. by using a conventional pixel patch matching algorithm—correlation based for example), the offsets between successive acquired images from the central image array—forming the “breadslice”—can be precisely located in 3D space. Once the data is acquired for a sequence of detected location planes, the volumetric data may be interpolated onto a regular 3D grade data set for easier subsequent manipulation. When using a freehand approach, the tracking image motion detection provides the system with scan speed (i.e. mm/s). Therefore, the system user interface will provide real-time feedback to the user—i.e. to “speed up” or “slow down” sweeping, optionally using a graphical “bull's eye” that the user keeps aligned to maintain the optimal scan speed.

However, it also appears that the total energy deposition to the target tissue influences the success of the treatment. Accordingly, in some embodiments, the ultrasound operator can sweep the transducer back and forth across the tissue target at a steady speed multiple times until the total time duration target has been met. For example, in early studies, it appears that a total time duration of about 15 minutes can be employed. However, the total time duration can be a function of: instantaneous ultrasound intensity and pulse “duty factor”—i.e. the proportion of elapsed time during which ultrasound is actually “on”. In some embodiments, as described hereinabove, the total time is between about 5 minutes and about 15 minutes (i.e., about 5, 6, 7, 8, 9, 10, 11, 12, 13, 14, or 15 minutes).

In some embodiments, a Verasonics programmable scanner (V-1 or Vantage (Verasonics Inc., Kirkland, Wash., United States of America) or another equivalent scanner can be programed to perform standard B-Mode imaging using any standard beamforming approach. Programming B-Mode, or other similar modes, for imaging falls within the knowledge of those “skilled in the art”, upon a review of the instant disclosure.

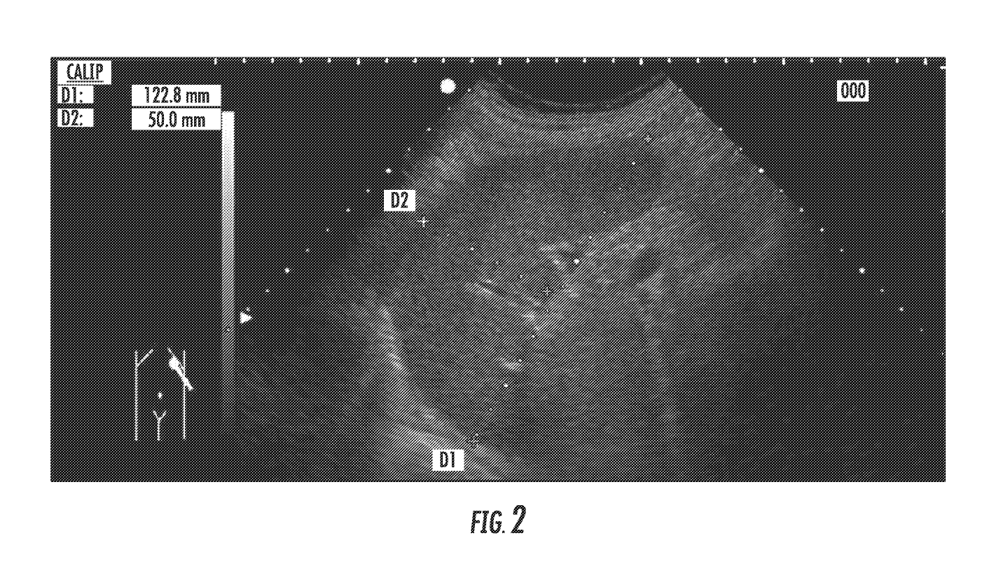

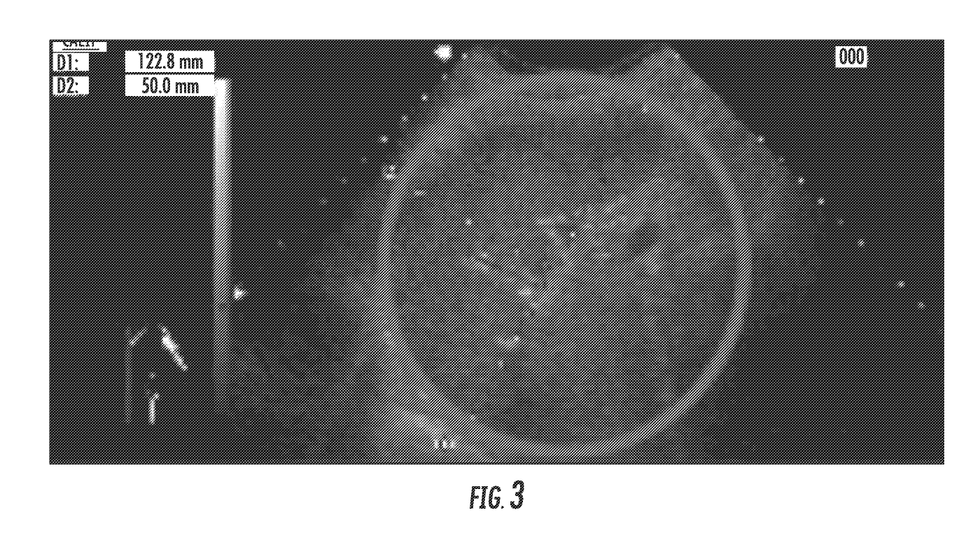

In some embodiments, therapeutic ultrasound can be performed, for example, using the same scanner (e.g., a Verasonics programmable scanner (Verasonics Inc., Kirkland, Wash., United States of America). The scanner can be used to create a user selectable menu of therapeutic protocols. As one example, the therapeutic ultrasound can be applied at about 3 MHz (range 1-10 MHz), Mechanical Index (MI)=1.2 (range 0.2 to 1.8), 6 (range 2-10) cycles, 0.2 ms (range 0.1 to 0.5 ms) intervals. A user programmed ROI can be selected that sets both the focal depth (middle of the ROI) and the width of the scan in terms of the number of lines required to encompass the ROI from side to side (laterally). See FIGS. 2 and 3, for example.

Accordingly, in some embodiments, user selectable options for the therapeutic ultrasound include: frequency, the number of pulse cycles per burst, the pulse amplitude (or MI), pulse interval, the focal depth, and the width of the focal zone. More particularly, in some embodiments, the frequency can be between about 1 MHz and about 10 MHz. In some embodiments, the frequency is about 1.7 MHz or about 3 MHz. In some embodiments, the number of cycles per burst can range from 2 to about 10 (e.g., 2, 3, 4, 5, 6, 7, 8, 9, or 10. In some embodiments, the number of cycles per burst can be 6. The MI can be between 0.2 to about 1.8 or 1.9 (where MI=1.9 is the FDA diagnostic ultrasound limit. In some embodiments, the MI can range from about 0.5 to about 1.9. In some embodiments, the MI is about 1 or higher, or such as about 1.2 or higher. In some embodiments, the pulse interval between pulses within burst patterns is between about 0.1 and about 0.5 ms. The focal depth can typically be the center of an ROI that encompasses the spleen. The focal zone width can be between about 10 and about 50 mm, e.g., encompassing the spleen in the ROI.

Additional user selectable options can include, but are not limited to, performing multiple repetitions per beam line. In B-Mode, successive line firings are typically on successive lateral locations, i.e., to sweep laterally across the field of view. However, in some embodiments, multiple firings along the same line can induce the desired therapeutic effect more efficiently. For example, in some embodiments, about 4 to about 10 beam repetitions can be performed at a single lateral location before stepping to the next location.

An aspect of an embodiment of the presently disclosed system (and method) provides for effecting therapeutic ultrasound, such as ultrasound-based protection from IRI, that can comprise, among other things, the sequence of:
a) imaging a target tissue region using ultrasound imaging;
b) identifying a volume region of interest (ROI) by highlighting a region on one of more ultrasound image frames as an ultrasound transducer is swept through a volume,
c) selecting a therapeutic ultrasound dose comprising a selected intensity, pulse length and/or pulse spacing; and
d) performing a volumetric sweep through the ROI systematically for a selected total time duration (for example, in the range 5 to 15 minutes).

Furthermore, an aspect of an embodiment of the presently disclosed system (and related method) can include, but is not limited to: a mechanical translation stage into which the ultrasound transducer can be placed to automatically effect the sweep; a transducer that is configured to track elevation motion to assure that a desired sweep velocity is used, such as by using “I-Beam”; a system that uses rates of image decorrelation to estimate rate of elevational motion and to provide feedback as to “too fast or too slow”, a transducer comprising a sector transducer array (i.e., that steers to +/−45 degrees), a transducer that comprises a curved linear transducer array, a transducer that comprises a two dimensional (2D) array that is configured to sweep through an entire target volume without physical translation; an ultrasound system configured to measure instantaneous sweep velocity and provide an audible or visual cue as to “too fast” or “too slow”, and/or a system that comprises an image library and/or the performance of image comparison/a similarity method to automatically identify the subject's spleen and thereby identify an optimal ROI that encompasses it.

Moreover, an aspect of an embodiment of the presently disclosed subject matter can be applicable to a variety of different modes of use. In some embodiments, a physician or other heath care professional can identify a subject for the presently disclosed methods via a non-imaging test (e.g., a blood test, such as a blood test performed post MI, etc.). In some embodiments, a physician or other health care professional can identify a subject based on the subject being scheduled to have surgery that can cause IRI. In some embodiments, the physician, other heath care provider and/or an ultrasound operator can identify the subject's spleen during imaging and immediately initiate a therapeutic ultrasound sweep. In some embodiments, a physician or other health care professional can initiate an imaging study and therapeutic sweep and direct successive sweeps on a daily, weekly or monthly basis.

Still yet, the presently disclosed system (and related method) can include variations of the methods described herein. For instance, in some embodiments, imaging is performed via a non-ultrasound based technique. Thus, in some embodiments, magnetic resonance imaging (MRI) or computerized tomography (CT) can be used to identify the spleen and then this form of imaging can be used to direct placement of therapeutic ultrasound (e.g., akin to magnetic resonance-guided focused ultrasound (MRgFUS). In some embodiments, contrast microbubbles can be infused prior to ultrasound to highlight and differentiate the spleen. In some embodiments, the ultrasound focal conditions can be modified, e.g., to time serial beams with progressively deeper foci (e.g., similar to multiple transmit focus in B-Mode ultrasound) or to spread focal depth beams (e.g., to use “axicon” beams).

It should be appreciated that a variety of ultrasound related systems and methods can be utilized as part of implementing or practicing aspects of the various embodiments of the presently disclosed subject matter.

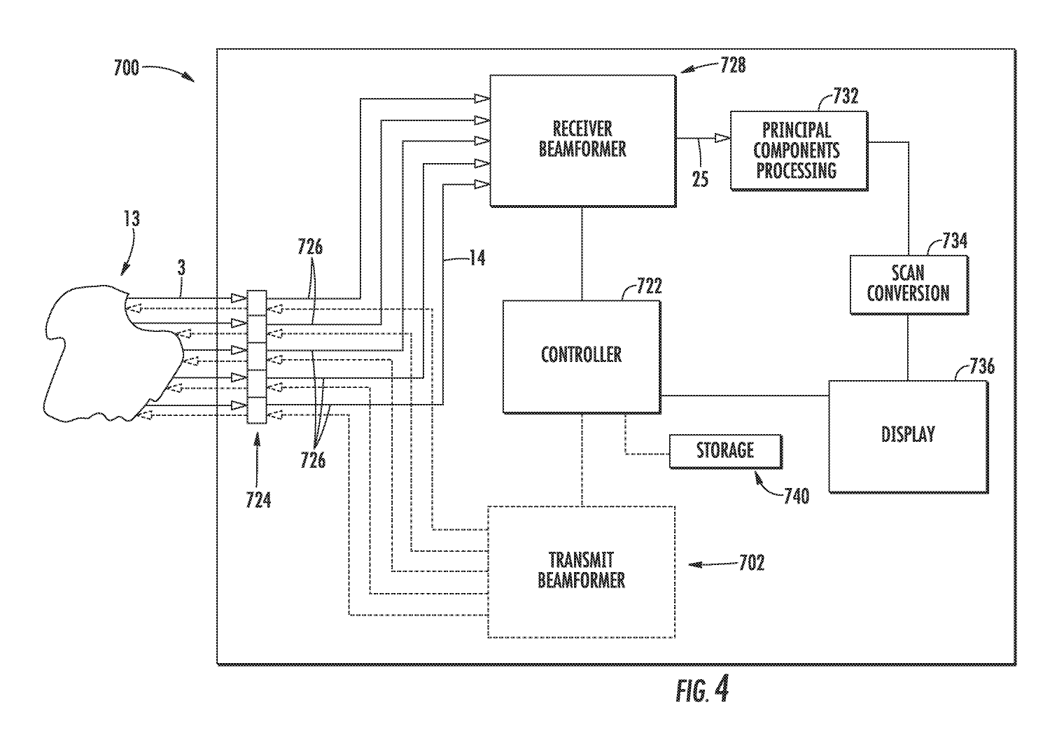

FIG. 4 is a basic, schematic representation of an ultrasound system 700 according to an aspect of an embodiment of the presently disclosed subject matter that is referred to in order to generally describe the operations of an ultrasound system to produce an image of an object 13 (e.g., a spleen inside a subject). System 700 can optionally include a transmit beamformer 702 which can include input thereto by controller 722 to send electrical instructions to array 724 (which can also be referred to as a transducer or a probe) as to the specifics of the ultrasonic waves to be emitted by array 724. Alternatively, system 700 can be a receive-only system and the emitted waves may be directed to the object 13 from an external source.

In either case, echoes 3 reflected by the object 13 (and surrounding environment) are received by array 724 and converted to electrical (e.g., radio frequency (RF)) signals 726 that are input to receive beamformer 728. Controller 722 can be external of the beamformer 728, as shown, or integrated therewith. Controller 722 can automatically and dynamically change the distances at which scan lines are performed (when a transmit beamformer 702 is included) and automatically and dynamically controls the receive beamformer 728 to receive signal data for scan lines at predetermined distances. Distance/depth is typically calculated assuming a constant speed of sound in tissue (e.g., 1540 m/s or as desired or required) and then time of flight is recorded such that the returning echoes have a known origination. The summed RF lines output by the receive beamformer 728 are input to a principal components processing module 732, which may be separate from and controlled by, or incorporated in controller 722. Principal components module 732 processes received echo data. The assembled output can be input into a scan converter module 734. The image formed within the scan converter 734 is displayed on display 736.

Controller 722 can have incorporated therein an input unit (e.g., comprising, for example, a keyboard, a mouse, a trackball, a tablet or a touch screen module) for the ultrasound operator to input control commands related to the operation of system 700. Control commands that can be input into controller 722 can include specifics of the ultrasonic waves emitted by transducer 724, such as, but not limited to, pulse frequency, pulse interval, etc. Alternatively, controller 722 and/or system 700 can be in communication with a machine 400 as describe further herein below.

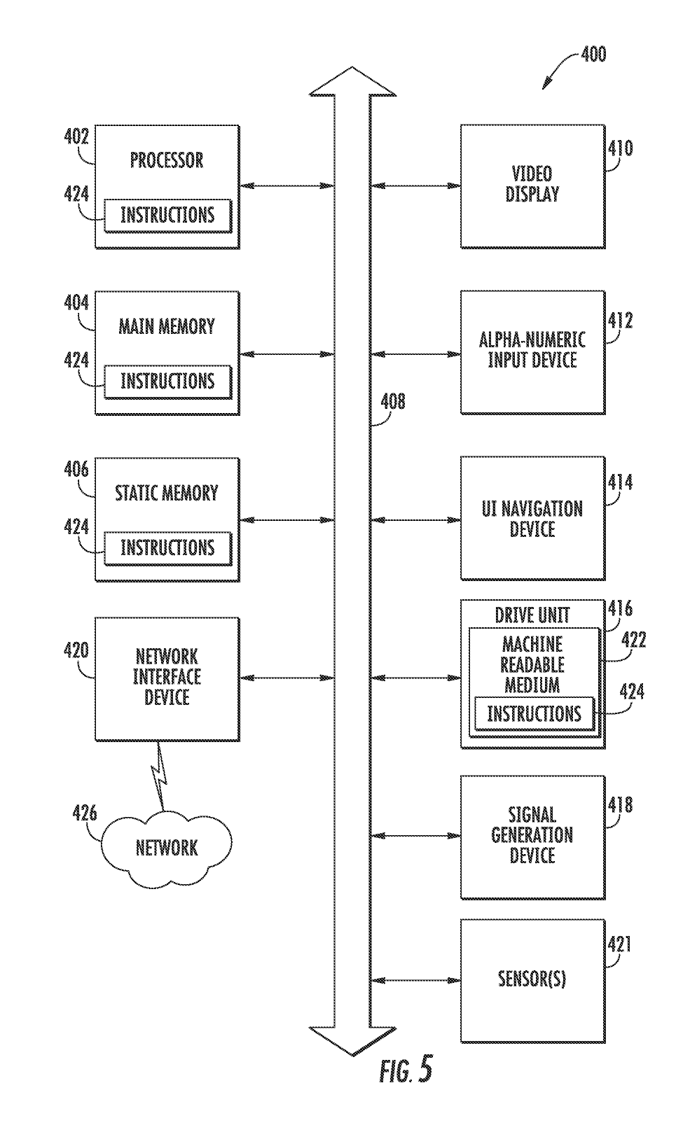

Transducer 724 is shown as an array in FIG. 5. Transducer 724 can be attached to the remainder of system 700 via a cable and be independently translatable, e.g., so that an ultrasound operator can manually translate transducer 724 over the skin surface of a subject to locate internal object 13 (e.g., spleen or another organ or tissue of interest). Alternatively, transducer 724 can be placed in an optional mechanical translation stage (e.g., controlled by controller 722) to automatically and systematically effect a sweep of particular volume of object 13. Transducer 724 can be a sector transducer array that steers to +/−45 degrees. Transducer 724 can be a curved linear array or a two dimensional (2D) array that can sweep an entire target volume of object 13 without physical translation. Further, although FIG. 5 has been described as an ultrasound system, it is noted that transducer 724 can alternatively be transducers for converting electrical energy to forms of energy other than ultrasound and vice versa, including, but not limited to radio waves (e.g., where system 700 is configured for RADAR), visible light, infrared, ultraviolet, and/or other forms of sonic energy waves, including, but not limited to SONAR, or some other arbitrary signal of arbitrary dimensions greater than one (such as, for example, a signal that is emitted by a target).

System 700 can optionally further include storage 740 which can store an image of object 13 (e.g., a previously acquired ultrasound image, or a computed tomography (CT) image acquired by an external CT scanner or a magnetic resonance (MR) image of object 13 obtained by an external magnetic resonance imaging (MRI) device. The previously acquired image can be a volume image of object 13 which includes volume information of object 13. If desired, storage 740 can include an image library database. The image library database can provide an image comparison/similarity algorithm to automatically identify spleen and, thereby, identify an optimal target region of interest (ROI) that encompasses the spleen.

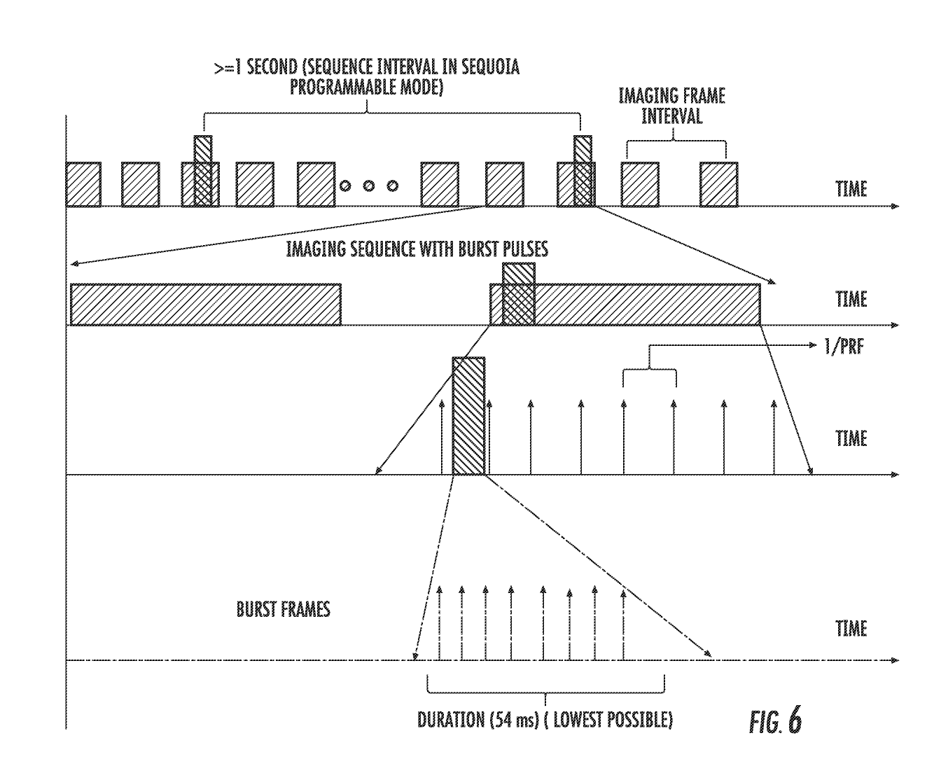

FIG. 6 is a block diagram illustrating an example of a machine upon which one or more aspects of embodiments of the presently disclosed subject matter can be implemented. FIG. 6 illustrates a block diagram of an example machine 400 upon which one or more embodiments (e.g., discussed methodologies) can be implemented (e.g., run).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In some embodiments, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Exemplary embodiments (e.g., apparatus, systems, or methods) can be implemented in digital electronic circuitry, in computer hardware, in firmware, in software, or in any combination thereof. Exemplary embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit410, input device 412 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

### III. EXAMPLES

### Example 1

### Initial US Studies in Pigs

An initial study to determine the effects of US pre-treatment on IRI caused by kidney ischemia was performed in a group of 15 pigs (5 controls). Kidney function after reperfusion was assessed by measuring plasma creatinine as a marker of impaired kidney function. The plasma creatinine concentration was reduced by an average of 47% in the US group vs. the control (no US) group using non-optimized instrumentation and methods.

### Example 2

### US Therapeutic Procedure Using Diagnostic US System

An ACUSON SEQUOIA™ 512 ultrasound machine (Siemens Medical Solutions USA, Inc., Mountain View, Calif., United States of America) was used to image and treat pigs (40-70 kg outbreed females) with US to determine the efficacy of US treatment on kidney IRI after 90 minutes of ischemia. To image the spleen, a 4V1 transducer was used and set at a frequency of 3 MHz (Plain, not Harmonic). Alpha cv CPS vascular can be chosen form the presets. Frame rate was reduced to as low as possible (e.g., 54 ms/19 Hz using the SEQUOIA™ machine). Focal depth can be adjusted using the keyboard to focus on the center of the spleen (approximately 40 mm). Burst duration was set for between 3 and 5 seconds, with 1 second between bursts. A stop watch was used to monitor time. Start burst was hit according to the protocol (every ten seconds for pig). Burst and imaging MI can be adjusted as needed. Both sagittal and transverse sections can be imaged.

Pigs can be continuously scanned along the long or short axes of the spleen, i.e., a sweep side to side (elevation) to encompass the target volume. The orientation of the transducer is not critical. The spleen was verified at autopsy to confirm that the target on the ultrasound system screen was the spleen.

The US procedure was also preformed using a Verasonics P4-2v transducer (Verasonics Inc., Kirkland, Wash., United States of America) transmitting at a frequency of 1.7 MHz during burst and at 3 MHz during imaging. A pulse sequence was programmed for a 3 second burst (with an interval between burst pulses of 6 ms and a burst A-line duration of 50 ms), with 1 second interval (steered through 128 angles (−45 to 45 degrees) due to small aperture of the transducer). During treatment the transducer was held at a specific location for 10 minutes. MI measured in water was 1.7 (in water) at 6 cm (focal distance was adjusted as spleen is more superficial). Treatment was manually stopped after 10 minutes by stopping the sequence.

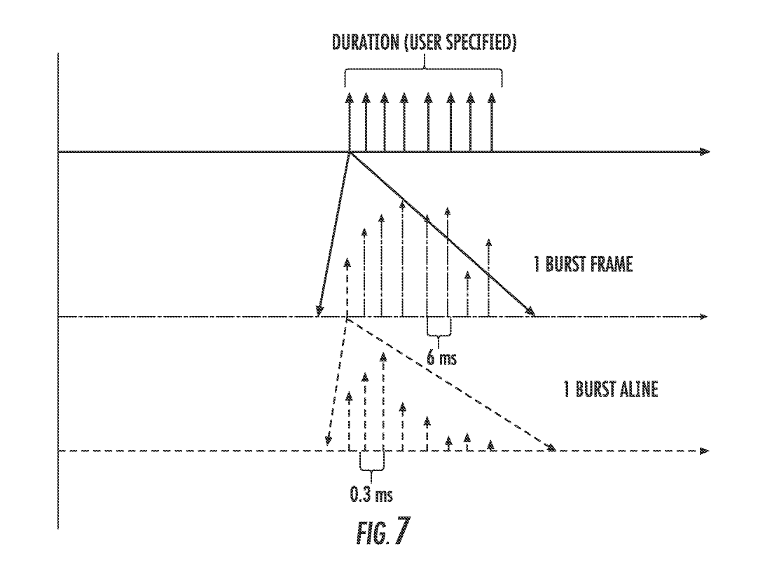

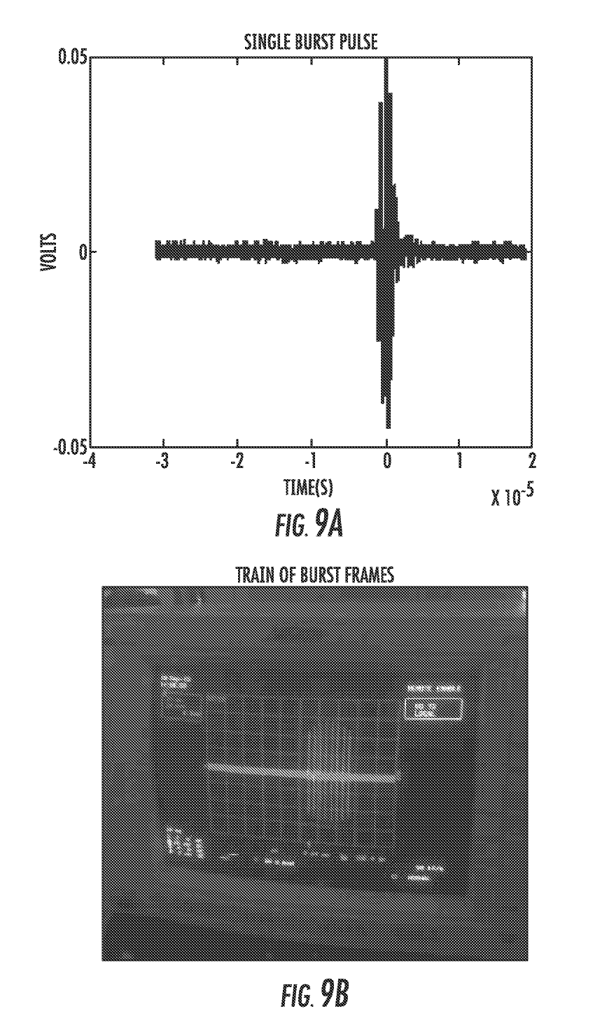

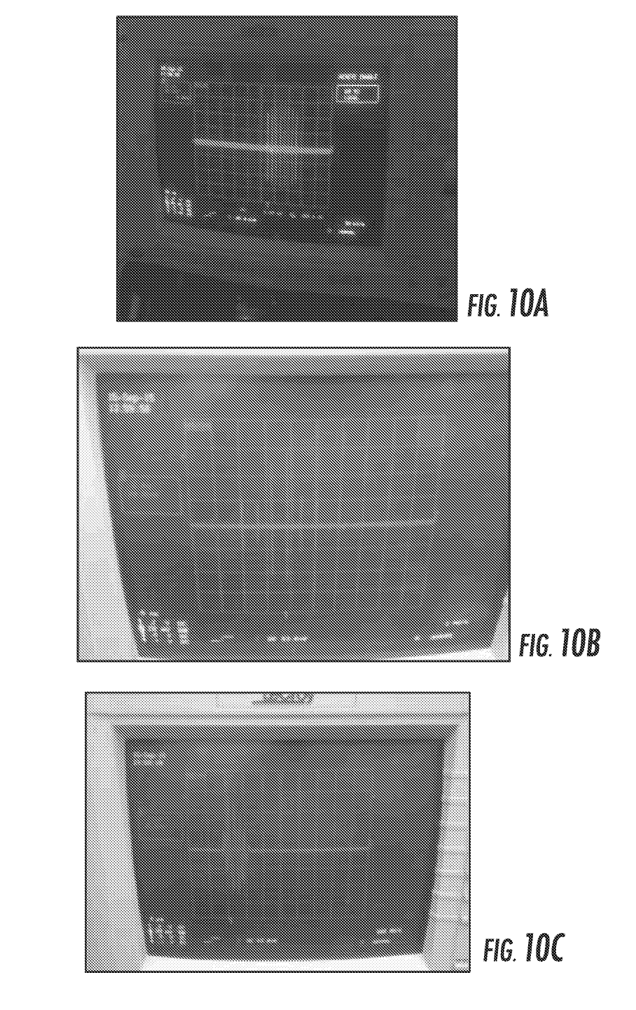

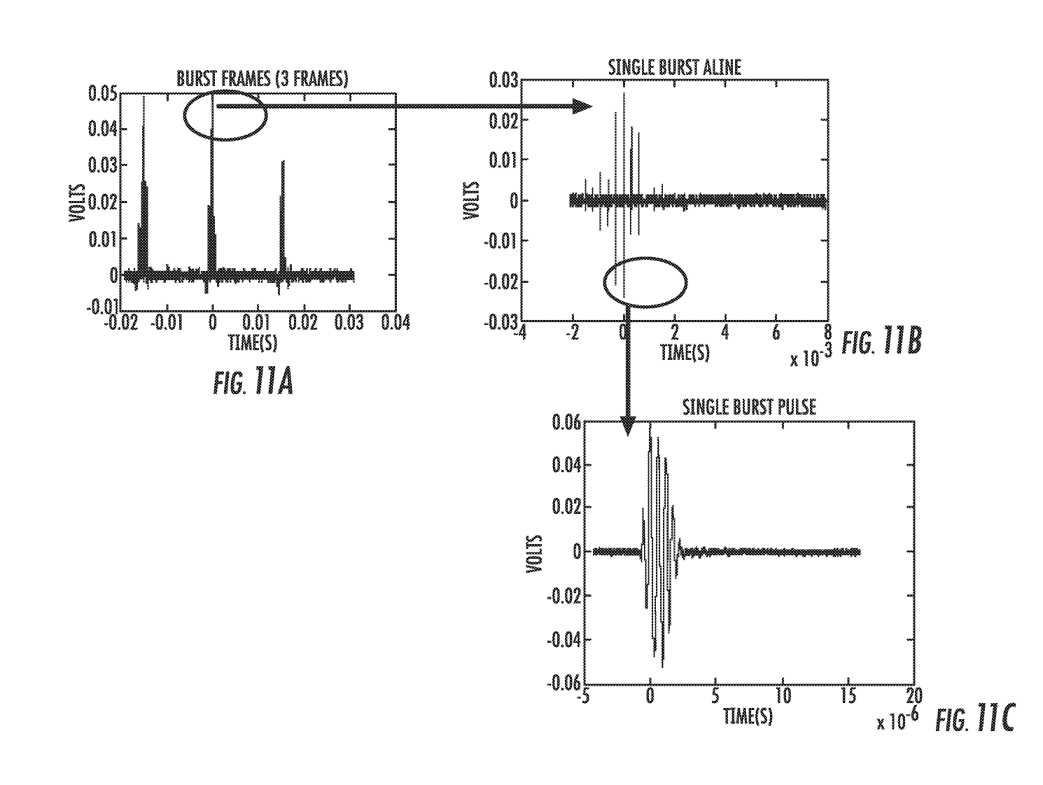

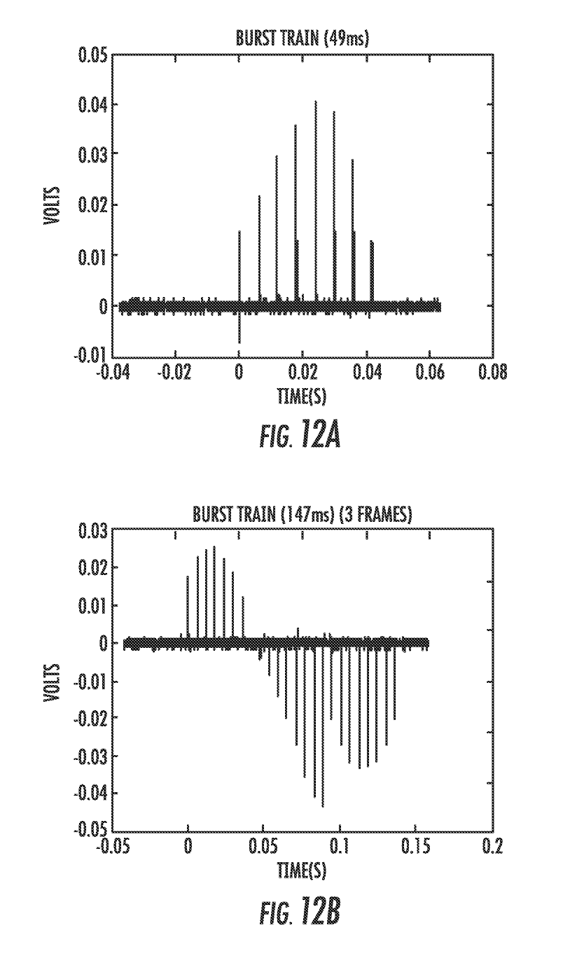

Additional details with regard to pulse sequences can be seen in FIGS. 6 and 7. A representative pulse sequence included burst pulses and imaging pulses. A set of single burst pulses can be referred to as a burst A-line, while a set of burst A-lines can be referred to as a Burst frame (e.g., 1 execution of a Burst sequence). See FIG. 7. The duration of the burst frame was programmed to be 54 ms. See FIG. 6. A single burst pulse comprises a 5 cycle pulse at 1.7 MHz. See FIGS. 9A, 10C, and 11C. FIGS. 10B and 11B show a single burst A-line with an interval between burst pulses of 0.3 ms. In FIG. 11B, the amplitude variation reflects off axis pulses. The interval between burst A-lines was 6 ms. See FIG. 10A. Multiple burst frames were performed sequentially to provide a burst train of a user specified duration, with a programmable interval (del T) between frames. See FIGS. 9B, 11A and 12B. Alternatively, a burst train can comprise a single burst frame. See FIGS. 10A and 12A. With the 54 ms burst frame duration, there is an upper bound of 9 A-lines in a burst frame and 20 pulses in 1 A-line. All A-lines and pulses are not picked up by the hydrophone because of off axis transmit.

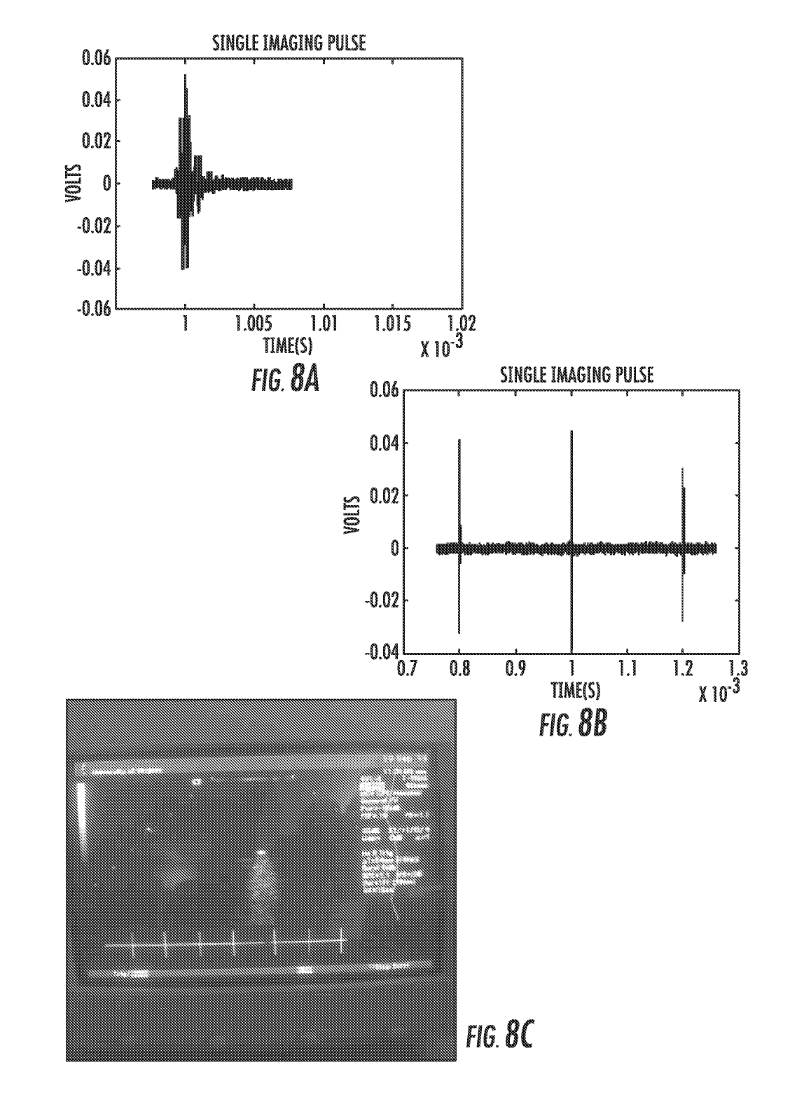

FIGS. 8A and 8B show typical imaging pulses and imaging pulse trains, while FIG. 8C shows a typical US image of a spleen from a single imaging pulse train with an imaging pulse at 2.8 MHz, an interval between pulses of 200 microseconds, a frame rate of 50 Hz and a focal depth of 60 mm.

### IV. REFERENCES

The following patents, applications and publications as listed below and throughout this document are hereby incorporated by reference in their entirety herein.

1. FUSF, http://www.fusfoundation.org/the-technology/manufacturers/manufacturers/list. 2015.

2. Gigliotti, J. C., et al., Ultrasound prevents renal ischemia-reperfusion injury by stimulating the splenic cholinergic anti-inflammatory pathway. J Am Soc Nephrol, 2013. 24(9): p. 1451-60.

3. Gigliotti, J. C., et al., Ultrasound Modulates the Splenic Neuroimmune Axis in Attenuating AKI. J Am Soc Nephrol, 2015.

4. Hossack, J. A., et al., Quantitative 3D Diagnostic Ultrasound Imaging Using a Modified Transducer Array and an Automated Image Tracking Technique. IEEE Transactions on Ultrasonics Ferroelectrics & Frequency Control, 2002. 49(8): p. 1029-1038.

5. U.S. Pat. No. 7,617,005 B2, Demarais, et al., “Methods and Apparatus for Thermally-Induced Renal Neuromodulation”, Nov. 10, 2009.

6. U.S. Pat. No. 7,717,948 B2, Demarais, et al., “Methods and Apparatus for Thermally-Induced Renal Neuromodulation”, May 18, 2010.

7. U.S. Pat. No. 9,186,198 B2, Demarais, et al., “Ultrasound Apparatuses for Thermally-Induced Renal Neuromodulation and Associated Systems and Methods”, Nov. 17, 2015.

The devices, systems, apparatuses, compositions, algorithms, machine readable medium, computer program products, and methods of various embodiments of the subject matter disclosed herein may utilize aspects disclosed in the following references, applications, publications and patents and which are hereby incorporated by reference herein in their entirety (and which are not admitted to be prior art with respect to the presently disclosed subject matter by inclusion in this section):

A. International Patent Application Publication No. WO 2003/075769, Walker, et al., An Intuitive Ultrasonic Imaging System and Related Method Thereof, Sep. 18, 2003.

B. U.S. Pat. No. 7,699,776, Walker, et al., Intuitive Ultrasonic Imaging System and Related Method Thereof, issued Apr. 20, 2010.

C. U.S. Patent Application Publication No. US 2010/0268086, Walker, et al., Intuitive Ultrasonic Imaging System and Related Method Thereof, Oct. 21, 2010.

D. U.S. Patent Application Publication No. US 2015/0011884, Walker, et al., Intuitive Ultrasonic Imaging System and Related Method Thereof, Jan. 8, 2015.

E. International Patent Application Serial No. PCT/US2004/000888, Blalock, et al., Ultrasonic Transducer Drive, filed Jan. 14, 2004.

F. U.S. Pat. No. 9,244,160, Blalock, et al., Ultrasonic Transducer Drive, issued Jan. 26, 2016.

G. U.S. patent application Ser. No. 15/005,565, Blalock, et al., Ultrasonic Transducer Drive, filed Jan. 25, 2016.

H. International Patent Application Serial No. PCT/US2004/000887, Walker, et al., Ultrasound Imaging Beam-Former Apparatus and Method, filed Jan. 14, 2004.

I. U.S. Patent Application Publication No. US2007/0016022, Blalock, et al., Ultrasound Imaging Beam-Former Apparatus and Method, Jan. 18, 2007.

J. U.S. Pat. No. 9,275,630, Blalock, et al., Ultrasound Imaging Beam-Former Apparatus and Method, issued Mar. 1, 2016.

K. International Patent Application Publication WO 04065978, Hossack, et al., Efficient Ultrasound System for Two-Dimensional C-Scan Imaging and Related Method Thereof, Aug. 5, 2004.

L. U.S. Pat. No. 7,402,136, Hossack, et al., Efficient Ultrasound System for Two-Dimensional C-Scan Imaging and Related Method Thereof, issued Jul. 22, 2008.

M. International Patent Application Publication No. WO 060412067, Hossack, et al., Efficient Architecture for 3D and Planar Ultrasonic Imaging—Synthetic Axial Acquisition and Method Thereof, Apr. 20, 2006.

N. U.S. Pat. No. 8,057,392, Hossack, et al., Efficient Architecture for 3D and Planar Ultrasonic Imaging—Synthetic Axial Acquisition and Method Thereof, issued Nov. 15, 2011.

O. U.S. Patent Application Publication No. US 2012/0029356, Hossack, et al., Efficient Architecture for 3D and Planar Ultrasonic Imaging—Synthetic Axial Acquisition and Method Thereof, Feb. 2, 2012.

P. U.S. Patent Application Publication No. US 2015/0025387, Hossack, et al., Efficient Architecture for 3D and Planar Ultrasonic Imaging—Synthetic Axial Acquisition and Method Thereof, Jan. 22, 2015.

Q. International Patent Application Publication No. WO 2008/154632, Garson, et al., System and Method for Combined ECG-Echo for Cardiac Diagnosis, Dec. 18, 2008.

R. U.S. Patent Application Publication No. US 2010/0168578, Garson, et al., System and Method for Combined ECG-Echo for Cardiac Diagnosis, Jul. 1, 2010.

S. U.S. Pat. No. 7,750,537, Hossack, et al., Hybrid Dual Layer Diagnostic Ultrasound Transducer Array, issued Jul. 6, 2010.

T. U.S. Pat. No. 8,093,782, Hossack, Specialized, High Performance, Ultrasound Transducer Substrates and Related Method Thereof, issued Jan. 10, 2012.

U. U.S. patent application Ser. No. 13/329,965, Hossack, Specialized, High Performance, Ultrasound Transducer Substrates and Related Method Thereof, filed Dec. 19, 2011.

V. International Patent Application Publication No. WO 2009/055720, Hossack, et al., System for Treatment and Imaging Using Ultrasonic Energy and Microbubbles and Related Method Thereof, Apr. 30, 2009.

W. U.S. Pat. No. 8,622,911, Hossack, et al., System for Treatment and Imaging Using Ultrasonic Energy and Microbubbles and Related Method Thereof, issued Jan. 7, 2014.

X. International Patent Application Publication No. WO 2011/011539, Hossack, et al., Systems and Methods for Ultrasound Imaging and Insonation of Microbubbles, Jan. 27, 2011.

Y. U.S. Pat. No. 9,237,898, Hossack, et al., Systems and Methods for Ultrasound Imaging and Insonation of Microbubbles, issued Jan. 19, 2016.

Z. U.S. Patent Application Publication No. US 2014/0142468, Hossack, et al., System for Treatment and Imaging Using Ultrasonic Energy and Microbubbles and Related Method Thereof, May 22, 2014.

AA. U.S. Patent Application Publication No. US 2015/0272601 A1, Hossack, et al., Method and Apparatus for Accelerated Disintegration of Blood Clot, Oct. 1, 2015.

BB. U.S. patent application Ser. No. 14/964,454, Hossack, et al., Method and Apparatus for Accelerated Disintegration of Blood Clot, filed Dec. 9, 2015.

CC. US. Patent Application Publication No. US 2010/0063399, Walker, et al., Front End Circuitry for Imaging Systems and Methods of Use, Mar. 11, 2010.

DD. International Patent Application Publication No. WO 2010/021709, Walker, et al., Front End Circuitry for Imaging Systems and Methods of Use, Feb. 25, 2010.

EE. U.S. Patent Application Publication No. US 2011/0137175, Hossack, et al., Tracked Ultrasound Vessel Imaging, Jun. 9, 2011.

FF. U.S. Pat. No. 9,002,080, Mauldin, et al., Singular Value Filter for Imaging or Detection, issued Apr. 7, 2015.

GG. International Patent Application Publication No. WO 2012/148985, Mauldin, Jr., et al., “Bone Surface Image Reconstruction Using Ultrasound”, Nov. 1, 2012.

HH. U.S. Patent Application Publication No. US 2014/0046186, Mauldin, Jr., et al., “Bone Surface Image Reconstruction Using Ultrasound”, Feb. 13, 2014.

II. International Patent Application Publication No. WO 2013/188625, Mauldin, Jr., et al., “Ultrasound Imaging of Specular-Reflecting Target”, Dec. 19, 2013.

JJ. U.S. Patent Application Publication No. US 2015/0133788, Mauldin, Jr., et al., “Ultrasound Imaging of Specular-Reflecting Target”, May 14, 2015.

KK. U.S. Patent Application Publication No. US 2015/0150534, Mauldin, Jr., et al.,” System and Method for Binding Dynamics of Targeted Microbubbles“, Jun. 4, 2015.

LL. International Patent Application No. PCTUS15/47374, Dixon, et al., “Method and Apparatus for Accelerated Disintegration of Blood Clot”, filed Aug. 28, 2015.

MM. International Patent Application No. PCT/US2015/064543, Hu, et al., “Multispectral Photoacoustic Microscopy Based on an Optical-acoustic Objective and Related Method Thereof”, filed Dec. 8, 2015.

Unless clearly specified to the contrary, there is no requirement for any particular described or illustrated activity or element, any particular sequence or such activities, any particular size, speed, material, duration, contour, dimension or frequency, or any particularly interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. It should be appreciated that aspects of the presently disclosed subject matter can have a variety of sizes, contours, shapes, compositions and materials as desired or required. In summary, while the presently disclosed subject matter has been described with respect to specific embodiments, many modifications, variations, alterations, substitutions, and equivalents will be apparent to those skilled in the art. The presently disclosed subject matter is not to be limited in scope by the specific embodiment described herein. Indeed, various modifications of the presently disclosed subject matter, in addition to those described herein, will be apparent to those of skill in the art from the foregoing description and accompanying drawings.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A method of protecting a subject in need thereof from ischemia/reperfusion injury (IRI) and/or mitigating in a subject an inflammatory condition, disease or disorder, the method comprising:
imaging a target tissue region within said subject, wherein the target tissue region comprises spleen tissue;
identifying a volume region of interest (ROI) in said target tissue; and
applying ultrasonic energy to the ROI, wherein applying the ultrasonic energy comprises emitting a sequence of ultrasonic pulses from an ultrasound transducer, the sequence of ultrasound pulses having predetermined frequency, mechanical index, pulse length, and pulse spacing, while performing a volumetric sweep through the ROI systematically for a selected total time duration of between about 3 minutes and about 15 minutes.

2. The method of claim 1, wherein the total time duration is about 10 minutes.

3. The method of claim 1 or claim 2, wherein the ultrasonic energy has a frequency of between about 1 and about 10 megahertz (MHz).

4. The method of any one of claims 1-3, wherein the sequence of ultrasound pulses comprises burst pulses having a frequency of about 1.7 MHz and imaging pulses having a frequency of about 3 MHz.

5. The method of any one of claims 1-4, wherein the ultrasonic energy is applied at a mechanical index ranging from about 0.5 to about 1.9.

6. The method of any one of claims 1-5, wherein the sequence of ultrasound pulses comprises a series of burst pulse sequences, optionally wherein the duration of each burst pulse sequence is between about 3 and about 5 seconds and the time interval between burst pulse sequences is about 1 second.

7. The method of any one of claims 1-6, wherein the ultrasonic energy “on” time, relative to the time between successive pulses, lies in the range of about 1% to about 10%.

8. The method of claim 6 or claim 7, wherein each burst pulse sequence comprises a series of pulse sub-sequences, optionally wherein each pulse sub-sequence comprises a series of pulse repetitions at a single lateral location.

9. The method of any one of claims 1-8, comprising protecting the subject from kidney IRI.

10. The method of any one of claims 1-9, wherein the subject is a human.

11. The method of any one of claims 1-10, wherein the ultrasonic energy is applied about 24 to 48 hours prior to the subject undergoing a surgery that will result in an ischemic event.

12. A system for effecting ultrasound-based protection from ischemia/reperfusion injury (IRI) and/or mitigating in a subject an inflammatory condition, disease or disorder, said system comprising:
an imaging device for imaging a target tissue region;
a transducer for performing a volumetric sweep through the target volume systematically for a selected total time duration and for applying ultrasonic energy to a volume region of interest (ROI), wherein applying the ultrasonic energy comprises emitting a sequence of ultrasonic pulses from the transducer, the sequence of ultrasound pulses having predetermined frequency, mechanical index, pulse length(s), and pulse spacing(s), while performing a volumetric sweep through the ROI systematically;
a processor for identifying a volume ROI by highlighting a region on one of more image frames as the transducer is swept through a volume; and
a controller for selecting a therapeutic ultrasound dose comprising a predetermined frequency, mechanical index, pulse length, and pulse spacing.

13. The system of claim 12, wherein the imaging device and the transducer are the same component.

14. The system of claim 12 or claim 13, wherein said transducer is placed in a mechanical translation stage to automatically effect the sweep.

15. The system of any one of claims 12-14, wherein said transducer is configured to track elevation motion to assure that a desired sweep velocity is used.

16. The system of any one of claims 12-15, wherein said transducer is a sector transducer array, optionally wherein it steers to +/−45 degrees.

17. The system of any one of claims 12-15, wherein said transducer is a curved linear transducer array.

18. The system of any one of claims 12-17, wherein said transducer comprises a 2D array that can sweep through an entire target volume without physical translation.

19. The system of any one of claims 12-18, wherein said selected total time duration ranges from about 3 minutes to about 15 minutes, optionally wherein said total time duration is about 10 minutes.

20. The system of any one of claims 12-19, wherein the ultrasonic energy has a frequency of between about 1 and about 10 megahertz (MHz).

21. The system of any one of claims 12-20, wherein the sequence of ultrasound pulses comprises burst pulses having a frequency of about 1.7 MHz and imaging pulses having a frequency of about 3 MHz.

22. The system of any one of claims 12-21, wherein the ultrasonic energy is applied at a mechanical index ranging from about 0.5 to about 1.9.

23. The system of any one of claims 12-22, wherein the sequence of ultrasound pulses comprises a series of burst pulse sequences, optionally wherein the duration of each burst pulse sequence is between about 3 and about 5 seconds and the time interval between burst pulse sequences is about 1 second.

24. The system of any one of claims 12-23, wherein the ultrasonic energy “on” time, relative to the time between successive pulses, lies in the range of about 1% to about 10%.

25. The system of claim 23 or claim 24, wherein each burst pulse sequence comprises a series of pulse sub-sequences, optionally wherein each pulse sub-sequence comprises a series of pulse repetitions at a single lateral location.

26. The system of any one of claims 12-25, wherein said system uses rates of image decorrelation to estimate rate of elevational motion and to provide feedback as to “too fast” or “too slow”.

27. The system of any one of claims 12-26, wherein said system measures instantaneous sweep velocity and provides and audible or visual cue as to “too fast” or “too slow”.

28. The system of any one of claims 12-27, wherein said system further comprises an image library database.

29. The system of claim 28, wherein said image library data base provides an image comparison/similarity algorithm that is used to automatically identify spleen and thereby identify an optimal ROI that encompasses it.

30. A non-transitory computer readable medium having stored thereon executable instructions that when executed by the processor of a computer control the computer to perform steps comprising:
imaging a target tissue region within said subject, wherein the target tissue region comprises spleen tissue;
identifying a volume region of interest (ROI) in said target tissue; and
applying ultrasonic energy to the ROI, wherein applying the ultrasonic energy comprises emitting a sequence of ultrasonic pulses from an ultrasound transducer, the sequence of ultrasound pulses having predetermined frequency, mechanical index, pulse length, and pulse spacing, while performing a volumetric sweep through the ROI systematically for a selected total time duration of between about 5 minutes and about 15 minutes.

31. The non-transitory computer readable medium of claim 30, wherein the total time duration is about 10 minutes.

32. The non-transitory computer readable medium of claim 30 or claim 31, wherein the ultrasonic energy has a frequency of between about 1 and about 10 megahertz (MHz).

33. The non-transitory computer readable medium of any one of claims 30-32, wherein the sequence of ultrasound pulses comprises burst pulses having a frequency of about 1.7 MHz and imaging pulses having a frequency of about 3 MHz.

34. The non-transitory computer readable medium of any one of claims 30-33, wherein the ultrasonic energy is applied at a mechanical index ranging from about 0.5 to about 1.9.

35. The non-transitory computer readable medium of any one of claims 30-34, wherein the sequence of ultrasound pulses comprises a series of burst pulse sequences, optionally wherein the duration of each burst pulse sequence is between about 3 and about 5 seconds and the time interval between burst pulse sequences is about 1 second.

36. The non-transitory computer readable medium of any one of claims 30-35, wherein the ultrasonic energy “on” time, relative to the time between successive pulses, lies in the range of about 1% to about 10%.

37. The non-transitory computer readable medium of claim 35 or claim 36, wherein each burst pulse sequence comprises a series of pulse sub-sequences, optionally wherein each pulse sub-sequence comprises a series of pulse repetitions at a single lateral location.

38. The non-transitory computer readable medium of any one of claims 30-37, wherein the executable instructions that when executed by the processor of a computer control the computer to interact with an image library database.

39. The non-transitory computer readable medium of any one of claims 30-38, wherein the executable instructions comprise an image comparison/similarity algorithm that is used to automatically identify spleen and thereby identify an optimal ROI that encompasses it.
